(19) United States
(12) Patent Application Publication
(10) Pub. No.: US 2009/0112018 A1
(43) Pub. Date: Apr. 30, 2009

(54) METHOD FOR PRODUCING POLYISOCYANATES

(75) Inventors: Martin Sesing, Waldsee (DE); Hartwig Voss, Frankenthal (DE); Eckhard Stroefer, Mannheim (DE); Thorsten Rohde, Mannheim (DE); Andreas Wolfert, Bad Rappenau (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/718,520

(22) PCT Filed: Oct. 22, 2005
(86) PCT No.: PCT/EP2005/011367

§ 371 Date: May 3, 2007

(30) Foreign Application Priority Data
Nov. 3, 2004 (DE) 102004053661.9

(51) Int. Cl. C07C 263/00

(52) U.S. Cl. 560/347

(57) ABSTRACT

The invention provides a process for preparing isocyanates by reacting the corresponding amines with phosgene in the presence of solvents, wherein compounds which form salt melts with hydrogen chloride are used as solvents.

The invention relates to a process for preparing polyisocyanates by reacting the corresponding amines with phosgene.

Polyisocyanates are produced in large quantities and serve mainly as starting materials for the production of polyurethanes. They are usually prepared by reacting the corresponding amines with phosgene.

Such processes are carried out on a large industrial scale and have been described widely, for example in Ullmanns Enzykiopädie der Technischen Chemie, or in the Kunststoffhandbuch, Volume 7 (Polyurethane), 3rd revised edition, Carl Hanser Verlag, Munich-Vienna, p. 76ff (1993).

In general, the continuous embodiment of this process is carried out in two stages. In the first phosgenation stage, the amine is reacted with phosgene to form carbamoyl chloride and hydrogen chloride and converted in a parallel reaction into amine hydrochloride. The reaction between amine and phosgene is very fast, strongly exothermic and proceeds even at very low temperatures. To minimize formation of byproducts and solids, amine and phosgene, if appropriate in admixture with an organic solvent, have to be mixed quickly. For this reason, the first phosgenation stage is generally carried out in a mixer, preferably a nozzle. The second phosgenation stage comprises both the decomposition of the carbamoyl chloride, which is usually present as a solid, to form the desired isocyanate and hydrogen chloride and the phosgenation of the amine hydrochloride to form carbamoyl chloride. The temperature of the second phosgenation stage is generally higher than that of the first. Many reactors have been developed for the second stage.

The hydrogen chloride formed in the reaction is usually removed from the reaction mixture very quickly in order to reduce the pressure in the reaction system and shift the equilibrium of the reaction in the direction of the isocyanates.

The reaction is generally carried out in the presence of solvents. Inert organic solvents such as toluene or chlorobenzenes are most frequently used. These solvents have to be separated off from the reaction mixture after the reaction.

A further possibility is the use of isocyanates as solvents. This is described, for example, in DE 1 192 641, DE 100 27 779 and DE 101 29 233. In this variant, the removal of the solvent after the phosgenation can be dispensed with. However, a disadvantage is that reaction of the polyisocyanates with the amines used to form ureas cannot be ruled out.

U.S. Pat. No. 5,136,086 describes the use of carboxylic esters as solvents for the reaction of the amines with phosgene. A disadvantage of this variant is that the solvents can react with the isocyanates.

An ever-present requirement in the preparation of isocyanates by reaction of the corresponding amines with phosgene is to achieve a reduction in the amount of phosgene present in the reaction system, also referred to as phosgene holdup. A further ever-present requirement in the preparation of polyisocyanates is to decrease the secondary reactions and thus to obtain a higher yield and products having improved quality.

It has now surprisingly been found that when solvents which, under the conditions of the reaction of the amines with phosgene, temporarily form salt melts with the hydrogen chloride formed are used, the space-time yield of the process increases and the secondary reactions are suppressed to a significant extent.

The invention accordingly provides a process for preparing isocyanates by reacting the corresponding amines with phosgene in the presence of solvents, wherein compounds which form salt melts with hydrogen chloride and from which the hydrogen chloride can be reversibly liberated again are used as solvents.

Possible solvents which temporarily form salt melts with the hydrogen chloride formed in the reaction are, in particular, ethers and polyethers.

The ethers can be acyclic or cyclic ethers. Examples are dioxane, tetrahydrofuran and glycol ethers such as diethylene glycol dimethyl ether (diglyme), ethylene glycol dimethyl ether (glyme). The solvents mentioned can be used alone or in admixture with other organic solvents. When other organic solvents are concomitantly used, the content of the solvents which are used according to the invention and form salt melts with hydrogen chloride should be at least 10% by weight, based on the total amount of solvents.

The conversion of the solvents into salts or salt melts can be effected prior to the reaction and the dissolution of the starting compounds. It is likewise possible for the starting compounds firstly to be dissolved in the solvent and the solvent then to be converted into the salt or the salt melt by addition of hydrogen chloride. The formation of the salts or salt melts is preferably effected temporarily by the hydrogen chloride formed in the reaction of the amines with phosgene.

The formation of the salts or salt melts is reversible, i.e. they can be converted back into solvent and hydrogen chloride. This can, for example, be effected by reducing the pressure and/or increasing the temperature.

At elevated pressure and low temperatures, the equilibrium is on the side of the salt melt. After leaving the reaction zone, the reaction system is depressurized and the solvent is thus separated from hydrogen chloride. The removal of the hydrogen chloride and the solvent from the reaction system can be effected, for example, in a flash vessel with a downstream evaporator. Here, the solvent and the hydrogen chloride are liberated from the salt or the salt melt and can be separated off from the isocyanate by distillation.

The solvent can also be separated from the hydrogen chloride by increasing the temperature. However, this embodiment is not preferred because of energy reasons.

The salt melt is more polar than the customary inert solvents and is better able to dissolve the solids occurring as intermediates in the phosgenation. The solids, in particular amine hydrochlorides and carbamoyl chlorides, can therefore react more quickly, which increases the space-time yield and reduces the problem of deposition of solids.

As described, the conversion of the solvents into salts or salt melts can occur either before or during the reaction of the amines with phosgene.

In one embodiment of the process of the invention, it is possible to dissolve the starting compounds in the solvent and react these solutions. The solvent is converted into the corresponding salt by the hydrogen chloride formed in the reaction.

In a further embodiment of the process of the invention, phosgene which is recirculated from the reaction and still contains hydrogen chloride is used for preparing the phosgene-containing solution. In this embodiment, the process step of separation of phosgene and hydrogen chloride after the preparation of the isocyanates can be dispensed with.

In a further embodiment, the solvent is brought into contact with hydrogen chloride before or after dissolution of the starting compounds and the salts or salt melts are formed in this way.

The process of the invention requires no additional starting materials for the reaction compared to the processes customary in industry. The process of the invention can thus be carried out without problems in existing plants.

The customary polyisocyanates produced on an industrial scale can be prepared by the process of the invention. These are, for example, the aromatic isocyanates TDI (tolylene diisocyanate) and MDI (methylenedi(phenyl isocyanate)), PMDI (polymethylenepolyphenylene polyisocyanate) and mixtures of MDI and PMDI (crude MDI) and also the aliphatic isocyanates HDI (hexamethylenedi(phenyl isocyanate)) and isophorone diisocyanate (IPDI).

The temperature range advantageous for the process of the invention depends, inter alia, on the type and amount of solvent and on the isocyanate to be prepared. In general, the temperature in the mixing unit is in the range from −20° C. to 300° C., preferably from 10° C. to 200° C. and particularly preferably from 80° C. to 150° C. The temperature in the reactor is generally in the range from 10° C. to 360° C. and preferably from 40° C. to 210° C. and particularly preferably from 80° C. to 150° C. Furthermore, the absolute pressure is generally in the range from 0.2 bar to 50 bar, preferably from 1 bar to 25 bar, particularly preferably from 3 to 17 bar.

The total residence time of the liquid in the mixing device and in the reactor is from 12 s to 20 minutes, preferably in the range from 36 s to 16 minutes and particularly preferably from 60 s to 12 minutes.

The molar ratio of phosgene used to amino groups is from 1:1 to 12:1, preferably from 1.1:1 to 6:1.

To carry out the process of the invention, the starting materials amine and phosgene are dissolved in the solvent used according to the invention: as an alternative, it is also possible to dissolve only the amine in the solvent. The two streams of amine in solution and phosgene, neat or in solution, are combined, preferably by means of a mixing nozzle. In a preferred embodiment, an axially symmetric mixing tube apparatus having axial introduction of amine and introduction of phosgene via two nonaxial annular gaps is used as mixing nozzle.

The amount of solvent used in the process of the Invention is generally from 10 to 1000% by weight, preferably from 50 to 500% by weight, more preferably from 100 to 400% by weight, based on the amount of amine used.

After the reaction, the mixture is preferably separated into isocyanate, solvent, phosgene and hydrogen chloride by means of rectification. Here, the decomposition of the salts or salt melts into the solvent and hydrogen chloride occurs, as described above. Small amounts of by-products which remain in the isocyanate can be separated from the desired isocyanate by means of additional rectification or crystallization.

The solvent and the phosgene can be recirculated and reused for the reaction. As described above, it is not necessary to recirculate HCl-free phosgene to the reaction.

Depending on the reaction conditions selected, the crude end product can further comprise inert solvent, carbamoyl chloride and/or phosgene and can be processed further by known methods, as described, for example, in WO 99/40059e. It can also be advantageous to pass the product through a heat exchanger when it is taken off.

The invention is illustrated by the following examples.

### EXAMPLE 1

### Comparison

### Preparation of MPI in Monochlorobenzene

2 g of MDAxHCl together with 98.03 g of monochlorobenzene were placed in a 400 ml pressure autoclave. 7.5 g of phosgene were added to this solution at 120° C., The phosgenation took place under the autogenous pressure of the reaction system at the reaction temperature.

### EXAMPLE 2

### According to the Invention

Preparation of MDI in 1,4-dioxane

2.0 g of MDAxHCI together with 98.95 g of 1,4-dioxane were placed in a 400 ml pressure autoclave. 6.5 g of phosgene were added to this solution at 120° C. The phosgenation took place under the autogenous pressure of the reaction system at the reaction temperature.

| | Example 1 | Example 2 | Example 1 | Example 2 |
|---|---|---|---|---|
| Reaction time | Conversion | Conversion | Pressure | Pressure |
| [s] | [%] | [%] | [bar] | [bar] |
| 0 | 0.0 | 0.0 | 0.76 | 3.33 |
| 29 | 20.9 | 32.4 | 2.72 | 3.34 |
| 57 | 28.5 | 47.9 | 2.93 | 3.36 |
| 122 | 40.4 | 61.2 | 3.12 | 3.38 |
| 179 | 46.0 | 67.6 | 3.25 | 3.40 |
| 304 | 52.4 | 76.7 | 3.46 | 3.41 |
| 485 | 58.6 | 82.7 | 3.66 | 3.42 |

It can be seen that a higher yield is achieved at virtually the same pressure when using the solvents employed according to the invention.

1. A process for preparing isocyanates by reacting the corresponding amines with phosgene in the presence of solvents, wherein compounds which form salt melts with hydrogen chloride are used as solvents.

2. The process according to claim 1, wherein ethers or polyethers are used as solvents.

3. The process according to claim 1, wherein the solvent is an acyclic and/or cyclic ether.

4. The process according to claim 1, wherein the solvent is selected from the group consisting of dioxane, tetrahydrofuran and glycol ethers such as diethylene glycol dimethyl ether, ethylene glycol dimethyl ether and/or polyoxymethylene dimethyl ether.

5. The process according to claim 1, wherein conversion of the solvents into salts or salt melts is effected prior to the reaction and the dissolution of the starting compounds.

6. The process according to claim 1, wherein the conversion of the solvents into salts or salt melts is effected at an intermediate stage by the hydrogen chloride formed in the reaction of the amines with phosgene.

7. The process according to claim 1, wherein the salt melt formed from the solvent and the hydrogen chloride formed in the reaction is redissociated into the solvent and the hydrogen chloride by reducing the pressure and/or increasing the temperature.
